(19) United States
(12) Patent Application Publication
(10) Pub. No.: US 2025/0012818 A1
(43) Pub. Date: Jan. 9, 2025

(54) Methodology to Measure Non-Structural Proteins and RNA Species from Human Hair

(71) Applicant: The Board of Trustees of the Leland Stanford Junior University, Stanford, CA (US)

(72) Inventors: Kanwaljeet S. Anand, Stanford, CA (US); Cynthia Rovnaghi, Scotts Valley, CA (US); Ryan Leib, Burlingame, CA (US)

(21) Appl. No.: 18/710,081

(22) PCT Filed: Nov. 10, 2022
(86) PCT No.: PCT/US2022/079619

§ 371 Date: May 14, 2024

Related U.S. Application Data
(60) Provisional application No. 63/279,458, filed on Nov. 15, 2021.

(51) Int. Cl. G01N 33/74; C12Q 1/6806; C12Q 1/6883; G01N 1/34

(52) U.S. Cl. CPC G01N 33/74; C12Q 1/6806; C12Q 1/6883; G01N 1/34; C12Q 2600/158

(57) ABSTRACT

Compositions and methods are provided for identifying and measuring biomarkers in hair. In particular, methods for extracting proteins and RNA species from hair, and for identifying and measuring hair biomarkers are disclosed. Serial segmentation of a hair sample can be used to assess sequential epochs of protein expression, metabolic regulation, and gene expression in the body across different time periods. Use of hair biomarkers provides a non-invasive, pain-free method for diagnosing diseases and identifying individuals at risk for poor health outcomes.

### BACKGROUND OF THE INVENTION

No methods are currently available for the holistic monitoring of long-term health, aging-related health decline, communicable or non-communicable diseases, or any other chronic health conditions. Critical gaps exist in indexing the health status of humans at all ages to reliably differentiate wellness versus illness, and pathogenesis versus salutogenesis. Multiple groups are trying to close these critical gaps by applying multiomic methodologies to serial blood samples that are obtained over several months or up to 2 years, or from the longitudinal sampling of other biofluids (urine, saliva, mucosal secretions, cerebrospinal fluid, or other tissue fluids). However, better noninvasive methods are needed to identify individuals at risk for poor health outcomes, enabling early intervention, avoidance of disease progression, and markedly reduced healthcare costs.

### SUMMARY OF THE INVENTION

Compositions and methods are provided for identifying and measuring biomarkers in hair. In particular, methods for extracting proteins and RNA from hair and identifying and measuring hair biomarkers are disclosed. Serial segmentation of a hair sample can be used to assess sequential epochs of protein expression, metabolic regulation, and gene expression in the body across different time periods. Use of hair biomarkers provides a non-invasive, pain-free method for diagnosing diseases and identifying individuals at risk for poor health outcomes.

In one aspect, a method of measuring biomarkers in a hair sample is provided, the method comprising: (a) obtaining a hair sample from a subject; (b) segmenting the hair sample to produce a sequential series of hair segments; (c) isolating RNA biomarkers and protein biomarkers from one or more of the hair segments; (d) removing structural hair proteins; and (e) measuring levels of the isolated RNA biomarkers and protein biomarkers.

In certain embodiments, isolating RNAs and protein biomarkers comprises: mixing the one or more hair segments with a monophasic solution of phenol and guanidinium isothiocyanate; adding chloroform to the monophasic solution; centrifuging the monophasic solution, wherein the monophasic solution is separated into an aqueous phase and an organic phase; isolating protein biomarkers from the organic phase; and isolating RNA biomarkers from the aqueous phase.

In certain embodiments, the method further comprises adding acetone to the organic phase to precipitate the protein biomarkers in the organic phase.

In certain embodiments, the method further comprises washing the precipitated protein biomarkers with a solution of guanidine hydrochloride.

In certain embodiments, the method further comprising washing the precipitated protein biomarkers with ethanol.

In certain embodiments, the hair segments are arranged in an order corresponding to their position along a length of hair to allow longitudinal sampling.

In certain embodiments, the hair sample has a weight of at least 20 mg. In some embodiments, the hair sample has a weight ranging from about 20 mg to about 200 mg. In some embodiments, the hair sample has a weight ranging from about 50 mg to about 100 mg.

In certain embodiments, the method is performed at room temperature.

In certain embodiments, the method is performed in under 3 hours. In certain embodiments, the analytical methods require up to 6 hours.

In certain embodiments, the RNA biomarkers are identified as messenger RNA (mRNA), or microRNA (miRNA), or transfer RNA (tRNA), or small nucleolar RNA (snoRNA), or ribosomal RNA (rRNA).

In certain embodiments, measuring the level of a RNA biomarker comprises performing a hybridization-based method, a polymerase chain reaction (PCR)-based method, or a nucleic acid sequencing method. For example the level of the RNA biomarker may be measured by performing microarray analysis, reverse transcriptase polymerase chain reaction (RT-PCR), Northern blotting, RNA-Seq, or serial analysis of gene expression (SAGE).

In certain embodiments, the protein biomarker is a non-structural hair protein such as, but not limited to, a peptide hormone (e.g., oxytocin), a receptor, a transcription factor, a transporter, an enzyme, or a growth factor.

In certain embodiments, measuring the level of a protein biomarker comprises performing mass spectrometry, or tandem mass spectrometry, an enzymatic or biochemical assay, liquid chromatography, NMR, an enzyme-linked immunosorbent assay (ELISA), a radioimmunoassay (RIA), an immunofluorescent assay (IFA), fluorescence-activated cell sorting (FACS), or a Western Blot.

In certain embodiments, a detergent is not used in any step of the method.

In certain embodiments, the method further comprises comparing said levels of biomarkers to reference levels of the biomarkers to determine a diagnosis or a prognosis for a subject. In some embodiments, the subject has not yet developed clinical symptoms. In other embodiments, the subject has developed clinical symptoms.

### DETAILED DESCRIPTION OF THE INVENTION

Before the compositions and methods for identifying and measuring hair biomarkers are described, it is to be understood that this invention is not limited to the particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a protein” includes a plurality of such proteins and reference to “the peptide” includes reference to one or more peptides and equivalents thereof, e.g., oligopeptides or polypeptides known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Biomarkers. The term “biomarker” as used herein refers to a compound, such as a protein, an RNA (e.g., an messenger RNA or a microRNA), a metabolite, or a metabolic byproduct which is differentially expressed or present at different concentrations, levels or frequencies in one hair sample compared to another, such as a hair sample from patients who have a disease or condition compared to a hair sample from healthy control subjects (i.e., subjects not having a disease or condition).

In some embodiments, the concentration or level of a biomarker is determined before and after the administration of a treatment to a patient. The degree of change in the concentration or level of a biomarker, or lack thereof, is interpreted as an indication of whether the treatment has the desired effect (e.g., halting or slowing progression of a disease, decreasing severity of a disease, ameliorating a symptom of the disease). In other words, the concentration or level of a biomarker is determined before and after the administration of the treatment to an individual, and the degree of change, or lack thereof, in the level is interpreted as an indication of whether the individual is “responsive” to the treatment.

A “reference level” or “reference value” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in hair samples (e.g., immunoassays (e.g., ELISA), mass spectrometry (e.g., LC-MS, GC-MS), tandem mass spectrometry, NMR, biochemical or enzymatic assays, PCR, microarray analysis, etc.), where the levels of biomarkers may differ based on the specific technique that is used.

A “similarity value” is a number that represents the degree of similarity between two things being compared. For example, a similarity value may be a number that indicates the overall similarity between a patient's biomarker profile using specific phenotype-related biomarkers and reference value ranges for the biomarkers in one or more control samples or a reference profile (e.g., the similarity to a biomarker expression profile for a disease or condition). The similarity value may be expressed as a similarity metric, such as a correlation coefficient, or may simply be expressed as the expression level difference, or the aggregate of the expression level differences, between levels of biomarkers in a patient sample and a control sample or reference expression profile.

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

Hair sample. The term “hair sample” with respect to an individual encompasses hair samples taken from the head or any other part of the body. Hair samples can be obtained by any suitable method such as by cutting hair with a scissors, razor, knife, or other device. The definition also includes samples that have been manipulated in any way after their procurement, such as by treatment with reagents; washed; or enriched for particular types of molecules, e.g., proteins (e.g., nonstructural hair proteins), nucleic acids (e.g., messenger RNAs, micro RNAs, transfer RNAs), etc.

Obtaining and assaying a sample. The term “assaying” is used herein to include the physical steps of manipulating a hair sample to generate data related to the hair sample. As will be readily understood by one of ordinary skill in the art, a hair sample must be “obtained” prior to assaying the sample. Thus, the term “assaying” implies that the hair sample has been obtained. The terms “obtained” or “obtaining” as used herein encompass the act of receiving an extracted or isolated hair sample. For example, a testing facility can “obtain” a hair sample in the mail (or via delivery, etc.) prior to assaying the hair sample. In some such cases, the hair sample was “extracted” or “isolated” from an individual by another party prior to mailing (i.e., delivery, transfer, etc.), and then “obtained” by the testing facility upon arrival of the hair sample. Thus, a testing facility can obtain the hair sample and then assay the hair sample, thereby producing data related to the hair sample.

The terms “obtained” or “obtaining” as used herein can also include the physical extraction of a hair sample from a subject. Accordingly, a hair sample can be removed from a subject (and thus “obtained”) by the same person or same entity that subsequently isolates biomarkers from the hair sample and/or assays the hair sample. When a hair sample is “obtained” from a first party or entity and then transferred (e.g., delivered, mailed, etc.) to a second party, the hair sample was “obtained” by the first party, and then subsequently “obtained” by the second party. The second party that obtains the hair sample may isolate biomarkers from the hair sample and/or assay the hair sample. Accordingly, in some embodiments, the step of obtaining does not comprise the step of isolating biomarkers from the hair sample and/or assaying the hair sample.

It will be understood by one of ordinary skill in the art that in some cases, it is convenient to wait until multiple hair samples have been obtained prior to assaying the hair samples. Accordingly, in some cases an isolated hair sample is stored until all appropriate samples have been obtained. One of ordinary skill in the art will understand how to appropriately store a hair sample, and any convenient method of storage may be used (e.g., refrigeration) that is appropriate for the particular hair sample. In some embodiments, a pre-treatment hair sample is assayed prior to obtaining a post-treatment hair sample. In some cases, a pre-treatment hair sample and a post-treatment hair sample are assayed in parallel. In some cases, multiple different hair samples are assayed in parallel. In some cases, hair samples are processed immediately or as soon as possible after they are obtained.

In some embodiments, the concentration (i.e., “level”), or expression level of a gene product, which may be an RNA, a protein, metabolite, etc., (which will be referenced herein as a biomarker), in a hair sample is measured (i.e., “determined”). By “expression level” (or “level”) it is meant the level of gene product (e.g., the absolute and/or normalized value determined for the RNA expression level of a biomarker or for the expression level of the encoded polypeptide, or the concentration of the protein in a hair sample). The term “gene product” or “expression product” are used herein to refer to the RNA transcription products (RNA transcripts, e.g., mRNA, an unspliced RNA, a splice variant mRNA, and/or a fragmented RNA) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The terms “determining”, “measuring”, “evaluating”, “assessing,” “assaying,” and “analyzing” are used interchangeably herein to refer to any form of measurement and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA or miRNA) of a particular biomarker. The level of expression can be expressed in arbitrary units associated with a particular assay (e.g., fluorescence units, e.g., mean fluorescence intensity (MFI)), or can be expressed as an absolute value with defined units (e.g., number of mRNA, miRNA, or protein molecules, concentration of RNA or protein, etc.). Additionally, the level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple hair samples from the same individual (e.g., hair samples taken at different points in time from the same individual or hair segments at different positions along the length of a hair that grew over time). This is because the units cancel when calculating a fold-change (i.e., determining a ratio) in the expression level from one hair sample to the next (e.g., hair samples taken at different points in time from the same individual or hair segments at different positions along the length of a hair that grew over time).

For measuring RNA levels, the amount or level of an RNA in the sample is determined, e.g., the level of an mRNA. In some instances, the expression level of one or more additional RNAs may also be measured, and the level of biomarker expression compared to the level of the one or more additional RNAs to provide a normalized value for the biomarker expression level. Any convenient protocol for evaluating RNA levels may be employed wherein the level of one or more RNAs in the assayed sample is determined.

A number of exemplary methods for measuring RNA (e.g., mRNA, miRNA) expression levels (e.g., expression level of a nucleic acid biomarker) in a sample are known by one of ordinary skill in the art, and any convenient method can be used. Exemplary methods include, but are not limited to: hybridization-based methods (e.g., Northern blotting, array hybridization (e.g., microarray); in situ hybridization; in situ hybridization followed by FACS; and the like)(Parker & Barnes, Methods in Molecular Biology 106:247-283 (1999)); RNase protection assays (Hod, Biotechniques 13:852-854 (1992)); PCR-based methods (e.g., reverse transcription PCR (RT-PCR), quantitative RT-PCR (qRT-PCR), real-time RT-PCR, etc.)(Weis et al., Trends in Genetics 8:263-264 (1992)); nucleic acid sequencing methods (e.g., Sanger sequencing, Next Generation sequencing (i.e., massive parallel high throughput sequencing, e.g., Illumina's reversible terminator method, Roche's pyrosequencing method (454), Life Technologies' sequencing by ligation (the SOLiD platform), Life Technologies' Ion Torrent platform, single molecule sequencing, etc.); and the like.

In some embodiments, the nucleic acids are assayed directly. In some embodiments, nucleic acids are amplified (e.g., by PCR) prior to assaying. As such, techniques such as polymerase chain reaction (PCR), reverse transcriptase PCR (RT-PCR), quantitative real time PCR (qRT-PCR), etc. can be used prior to the hybridization methods and/or the sequencing methods discussed above.

For measuring RNA levels, the starting material is typically total RNA or poly A+RNA isolated from a hair sample (e.g., hair segment). Methods for RNA extraction from a hair sample using guanidinium isothiocyanate (e.g., TRlzol® reagent) are described in Example 1.

A variety of different manners of measuring RNA levels are known in the art, e.g. as employed in the field of differential gene expression analysis. One representative and convenient type of protocol for measuring RNA (e.g., mRNA or miRNA) levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan® RT-PCR, MassARRAY® System, BeadArray® technology, and Luminex technology; and those that rely upon hybridization of probes to filters, e.g. Northern blotting and in situ hybridization.

Examples of some of the nucleic acid sequencing methods listed above are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps.

For measuring protein levels, the amount or level of a protein in the hair sample is determined. In some cases, the polypeptide comprises a post-translational modification (e.g., phosphorylation) associated with regulation of activity of the protein such as by a signaling cascade, wherein the modified protein is the biomarker, and the amount of the modified protein is therefore measured. In some embodiments, concentration is a relative value measured by comparing the level of one protein relative to another protein. In other embodiments the concentration is an absolute measurement of weight/volume or weight/weight.

In some cases, the structural hair proteins are removed from the hair sample (e.g., as described in Example 1.) prior to isolating and/or measuring the levels of non-structural hair proteins, RNA, or other hair biomarkers. Non-structural proteins may be isolated from a hair sample as described in Example 1.

In some instances, a biomarker concentration may be compared to the level of one or more additional proteins to provide a normalized value for the biomarker concentration. Any convenient protocol for evaluating protein levels may be employed wherein the level of one or more proteins in the assayed sample is determined.

While a variety of different manners of assaying for protein levels are known to one of ordinary skill in the art and any convenient method may be used, one representative and convenient type of protocol for assaying protein levels is ELISA, an antibody-based method. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be modified in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed. Representative exemplary methods include but are not limited to antibody-based methods (e.g., Western blotting, proteomic arrays, xMAP microsphere technology (e.g., Luminex technology), immunohistochemistry, flow cytometry, and the like) as well as non-antibody-based methods (e.g., mass spectrometry or tandem mass spectrometry).

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

### Additional Terms.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with a disease or condition) as well as those in which prevention is desired (e.g., those with a genetic predisposition to developing a disease or a condition, those with increased susceptibility to developing a disease or condition because of an environmental exposure to a toxic agent, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

A “therapeutically effective dose” or “therapeutic dose” is an amount sufficient to effect desired clinical results (i.e., achieve therapeutic efficacy). A therapeutically effective dose can be administered in one or more administrations.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “antibody” encompasses monoclonal antibodies, polyclonal antibodies, as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); bispecific antibodies, bispecific T cell engager antibodies (BiTE), trispecific antibodies, and other multispecific antibodies (see, e.g., Fan et al. (2015) J. Hematol. Oncol. 8:130, Krishnamurthy et al. (2018) Pharmacol Ther. 185:122-134), F(ab′)₂ and F(ab) fragments; Fᵥ molecules (noncovalent heterodimers, see, for example, Inbar et al. (1972) Proc Nat/Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen (e.g., biomarker) refers to a binding reaction that is determinative of the presence of the antigen in a heterogeneous population of proteins and other biologics. Thus, under designated immunoassay conditions, the specified antibodies bind to a particular antigen at least two times over the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of hair sample(s) was used and/or how it was used, the technique used to assay the sample, the results of the assay (e.g., the level of the biomarker as measured and/or the fold-change of a biomarker level over time or in a post-treatment assay compared to a pre-treatment assay), the assessment as to whether the individual is determined to have a disease or condition or at risk of developing a disease or condition, a recommendation for treatment, and/or to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

Isolating Biomarkers from a Hair Sample and Diagnostic Methods

Use of hair biomarkers provides a non-invasive, pain-free method for diagnosing diseases and identifying individuals at risk for poor health outcomes. A hair sample comprising hair biomarkers may be obtained from a subject by any suitable method. The hair sample is typically taken from the head of the subject but may be taken from any part of the body where hair grows. Hair samples can be obtained, for example, by cutting hair with a scissors, razor, knife, or other device. A “control” sample, as used herein, refers to a hair sample from a subject that is not diseased. That is, a control sample is obtained from a normal or healthy subject, or an individual known to not have a particular disease or condition.

In certain embodiments, the hair sample has a weight of at least 20 mg, at least 30 mg, at least 40 mg, at least 50 mg, at least 75 g, at least 100 mg, at least 150 mg, at least 200 mg, or more. In some embodiments, the hair sample has a weight ranging from about 20 mg to about 200 mg, including any weight within this range such as 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg. In some embodiments, the hair sample has a weight ranging from about 50 mg to about 100 mg.

Serial segmentation of a hair sample can be used to assess sequential epochs of protein expression, metabolic regulation, and/or gene expression in the body across different time periods. For example, a hair sample can be segmented to produce a sequential series of hair segments. In certain embodiments, the hair segments are labeled or arranged in an order corresponding to their position along a length of hair to allow longitudinal sampling.

In some embodiments, a hair sample is segmented into at least 2, at least 3, at least 4, at least 5, at least 6, at least 7, at least 10, at least 15, or at least 20 or more segments. In some embodiments, a hair sample is segmented into 2 to 100 segments, including any number of segments in this range such as 2, 4, 6, 8, 10, 12, 14, 16, 18, 20, 25, 30, 35, 40, 45, 50, 55, 60, 65, 70, 75, 80, 85, 90, 95, or 100 segments.

Methods of extracting proteins and RNA from hair and identifying and measuring hair biomarkers are disclosed in Example 1. In some embodiments, the method for extracting proteins and RNA from a hair sample comprises: mixing one or more hair segments with a monophasic solution of phenol and guanidinium isothiocyanate; adding chloroform to the monophasic solution; centrifuging the monophasic solution, wherein the monophasic solution is separated into an aqueous phase and an organic phase; isolating proteins from the organic phase; and isolating RNAs from the aqueous phase. In certain embodiments, the method further comprises adding acetone to the organic phase to precipitate the proteins in the organic phase. In certain embodiments, the method further comprises washing the precipitated proteins with a solution of guanidine hydrochloride. In certain embodiments, the method further comprising washing the precipitated proteins with ethanol. All steps of the method may be performed at room temperature.

Hair biomarkers may include RNAs, proteins, or metabolites. When analyzing the levels of biomarkers in a hair sample from a subject, the reference value ranges used for comparison can represent the levels of one or more biomarkers in a hair sample from one or more subjects without a disease (i.e., normal or healthy control). Alternatively, the reference values can represent the levels of one or more biomarkers from one or more subjects with a disease, wherein similarity to the reference value ranges indicates the subject has the disease.

The methods described herein may be used to determine an appropriate treatment regimen for a patient and, in particular, whether a patient should be treated for a disease or if preventive measures should be taken to avoid developing a disease. The subject methods may also be used for assaying pre-treatment and post-treatment hair samples obtained from an individual to determine whether the individual is responsive or not responsive to a treatment.

In some cases, combinations of biomarkers are used in the subject methods. In some such cases, the levels of all measured biomarkers must change (as described above) in order for the diagnosis to be made. In some embodiments, only some biomarkers are used in the methods described herein. For example, a single biomarker, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, or 19 biomarkers can be used in any combination. In other embodiments, all the biomarkers are used. The quantitative values may be combined in linear or non-linear fashion to calculate one or more risk scores for a disease for the individual.

The level of a biomarker in a pre-treatment hair sample can be referred to as a “pre-treatment value” because the first hair sample is isolated from the individual prior to the administration of the therapy (i.e., “pre-treatment”). The level of a biomarker in the pre-treatment hair sample can also be referred to as a “baseline value” because this value is the value to which “post-treatment” values are compared. In some cases, the baseline value (i.e., “pre-treatment value”) is determined by determining the level of a biomarker in multiple (i.e., more than one, e.g., two or more, three or more, for or more, five or more, etc.) pre-treatment hair samples. In some cases, the multiple pre-treatment hair samples are isolated from an individual at different time points in order to assess natural fluctuations in biomarker levels prior to treatment. As such, in some cases, one or more (e.g., two or more, three or more, for or more, five or more, etc.) pre-treatment hair samples are isolated from the individual. In some embodiments, all of the pre-treatment hair samples will be the same type of hair sample. In some cases, two or more pre-treatment hair samples are pooled prior to determining the level of the biomarker in the hair samples. In some cases, the level of the biomarker is determined separately for two or more pre-treatment hair samples and a “pre-treatment value” is calculated by averaging the separate measurements.

A post-treatment hair sample is isolated from an individual after the administration of a therapy. Thus, the level of a biomarker in a post-treatment sample can be referred to as a “post-treatment value”. In some embodiments, the level of a biomarker is measured in additional post-treatment hair samples (e.g., a second, third, fourth, fifth, etc. post-treatment hair sample). Because additional post-treatment hair samples are isolated from the individual after the administration of a treatment, the levels of a biomarker in the additional hair samples can also be referred to as “post-treatment values.”

The term “responsive” as used herein means that the treatment is having the desired effect such as halting or slowing progression of a disease, decreasing severity of a disease, or ameliorating a symptom of the disease. When the individual does not improve in response to the treatment, it may be desirable to seek a different therapy or treatment regime for the individual.

The determination that an individual has a disease or condition or is at risk of developing a disease or condition is an active clinical application of the correlation between levels of a biomarker and the disease or condition. For example, “determining” requires the active step of reviewing the data, which is produced during the active assaying step(s), and resolving whether an individual does or does not have a disease, or at risk of developing a disease, or is responding or not responding to a therapy for treatment of a disease. Additionally, in some cases, a decision is made to proceed with the current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a therapy) is to continue. If the current course of treatment is not effective in treating a disease, the treatment may be altered. “Altering therapy” is used herein to mean “discontinuing therapy” or “changing the therapy” (e.g., changing the type of treatment, changing the particular dose and/or frequency of administration of medication, e.g., increasing the dose and/or frequency). In some cases, therapy can be altered until the individual is deemed to be responsive. In some embodiments, altering therapy means changing which type of treatment is administered, discontinuing a particular treatment altogether, etc.

In other words, the level of one or more biomarkers may be monitored in order to determine when to continue therapy and/or when to alter therapy. As such, a post-treatment hair sample can be isolated after any of the administrations and the hair sample can be assayed to determine the level of a biomarker. Accordingly, the subject methods can be used to determine whether an individual being treated a disease is responsive or is maintaining responsiveness to a treatment.

The therapy can be administered to an individual any time after a pre-treatment hair sample is isolated from the individual, but it is preferable for the therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment hair sample is isolated (or, when multiple pre-treatment hair samples are isolated, after the final pre-treatment hair sample is isolated).

In some embodiments, the subject methods include providing an analysis indicating whether the individual is determined to have a disease or condition or is at risk of developing a disease or condition. The analysis may further provide an analysis of whether an individual is responsive or not responsive to a treatment, or whether the individual is determined to be maintaining responsiveness or not maintaining responsiveness to a treatment for a disease. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

### Detecting and Measuring Biomarkers

It is understood that the biomarkers in a hair sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites or metabolic byproducts) that are indicative of the expression level of the biomarker. The methods for measuring biomarkers in a sample have many applications. For example, one or more biomarkers can be measured to aid in evaluating the risk of a subject of developing a disease or condition and determining the appropriate treatment for a subject, as well as monitoring responses of a subject to treatment.

In some embodiments, the amount or level in the hair sample of one or more proteins/polypeptides encoded by a gene of interest is determined. Any convenient protocol for evaluating protein levels may be employed where the level of one or more proteins in the assayed sample is determined. For antibody-based methods of protein level determination, any convenient antibody can be used that specifically binds to the intended biomarker (e.g., hair non-structural protein, mRNA, microRNA). The terms “specifically binds” or “specific binding” as used herein refer to preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a K_{d} (dissociation constant) of 10⁻⁵ M or less (e.g., 10⁻⁶ M or less, 10-7 M or less, 10⁻⁸ M or less, 10-9 M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). By “Affinity” it is meant the strength of binding, increased binding affinity being correlated with a lower K_{d}.

While a variety of different manners of assaying for protein levels are known in the art, one representative and convenient type of protocol for assaying protein levels is the enzyme-linked immunosorbent assay (ELISA). In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer. The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed and any convenient method may be used. Representative examples known to one of ordinary skill in the art include but are not limited to other immunoassay techniques such as radioimmunoassays (RIA), sandwich immunoassays, fluorescent immunoassays, enzyme multiplied immunoassay technique (EMIT), capillary electrophoresis immunoassays (CEIA), and immunoprecipitation assays; mass spectrometry, or tandem mass spectrometry, proteomic arrays, xMAP microsphere technology, western blotting, and immunohistochemistry. As with ELISAs and immunohistochemistry, antibodies (e.g., monoclonal antibodies) that specifically bind the polypeptides encoded by the genes of interest are used in such methods.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The hair biomarkers can be added to a fluid that is introduced to the sensor either by submerging the electrodes in the fluid or by adding the fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

For measuring protein activity levels, the amount or level of protein activity in the hair sample of one or more proteins/polypeptides encoded by a gene of interest is determined.

In other embodiments, the amount or level in the hair sample of one or more RNAs encoded by a gene of interest is determined. Any convenient method for measuring mRNA levels in a sample may be used, e.g. hybridization-based methods, e.g. northern blotting and in situ hybridization (Parker & Barnes, Methods in Molecular Biology 106:247-283 (1999)), RNase protection assays (Hod, Biotechniques 13:852-854 (1992)), and PCR-based methods (e.g. reverse transcription PCR (RT-PCR) (Weis et al., Trends in Genetics 8:263-264 (1992)). Alternatively, any convenient method for measuring protein levels in a sample may be used, e.g. antibody-based methods, e.g. immunoassays, e.g., enzyme-linked immunosorbent assays (ELISAs), immunohistochemistry, and flow cytometry (FACS).

For measuring levels of mRNAs, microRNAs, or other RNAs, the starting material may be total RNA, i.e., unfractionated RNA, or poly A+RNA isolated from a hair sample. General methods for RNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

The RNA levels (e.g., mRNA or microRNA levels) may be measured by any convenient method. Examples of methods for measuring mRNA, microRNA, or other RNA levels may be found in, e.g., the field of differential gene expression analysis. One representative and convenient type of protocol for measuring RNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be employed in the subject methods includes that described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Additionally or alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan, RT-PCR, MassARRAY System, BeadArray technology, and Luminex technology; and those that rely upon hybridization of probes to filters, e.g. Northern blotting and in situ hybridization. Serial Analysis Gene Expression (SAGE) can also be used to determine RNA abundances in a cell sample. See, e.g., Velculescu et al., 1995, Science 270:484-7; Carulli, et al., 1998, Journal of Cellular Biochemistry Supplements 30/31:286-96; herein incorporated by reference in their entireties. SAGE analysis does not require a special device for detection, can be used for simultaneously detecting the expression of large numbers of transcription products.

The resultant data provides information regarding expression, amount, and/or activity for each of the biomarkers that have been measured, wherein the information is in terms of whether or not the biomarker is present (e.g. expressed) and at what level, and wherein the data may be both qualitative and quantitative.

### Data Analysis

In some embodiments, one or more pattern recognition methods can be used in analyzing the data for biomarker levels. The quantitative values may be combined in linear or non-linear fashion to calculate one or more risk scores for a disease or condition for an individual. In some embodiments, measurements for a hair biomarker or combinations of hair biomarkers are formulated into linear or non-linear models or algorithms (e.g., a ‘biomarker signature’) and converted into a likelihood score. This likelihood score may indicate the probability that a hair sample is from a patient who exhibits no evidence of disease or who exhibits evidence of a disease or a risk of developing a disease. The models and/or algorithms can be provided in machine readable format, and may be used to correlate biomarker levels or a biomarker profile with a disease state, and/or to designate a treatment modality for a patient or class of patients.

Analyzing the levels of a plurality of hair biomarkers may comprise the use of an algorithm or classifier. In some embodiments, a machine learning algorithm is used to classify a patient as having a disease or a risk of developing a disease. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Kits

Any of the compositions described herein may be provided in a kit. The subject kits may include agents for isolating protein and RNA biomarkers from a hair sample according to the methods described herein. For example, the kit may include phenol, guanidinium isothiocyanate, chloroform, acetone, ethanol, and the like. The kit may further include one or more containers for collecting hair samples and a tool (e.g., scissors, razor, or knife) for segmenting a hair sample.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-23 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A method of measuring one or more biomarkers in a hair sample, the method comprising:
(a) obtaining a hair sample from a subject;
(b) segmenting the hair sample to produce a sequential series of hair segments;
(c) isolating RNA biomarkers and protein biomarkers from one or more of the hair segments;
(d) removing structural hair proteins; and
(e) measuring levels of the isolated RNA biomarkers and protein biomarkers.

2. The method of aspect 1, wherein said isolating RNA biomarkers and protein biomarkers comprises:
(a) mixing the one or more hair segments with a monophasic solution of phenol and guanidinium isothiocyanate;
(b) adding chloroform to the monophasic solution;
(c) centrifuging the monophasic solution, wherein the monophasic solution is separated into an aqueous phase and an organic phase;
(d) isolating protein biomarkers from the organic phase; and
(e) isolating RNA biomarkers from the aqueous phase.

3. The method of aspect 2, further comprising adding acetone to the organic phase to precipitate the protein biomarkers in the organic phase.

4. The method of aspect 3, further comprising washing the precipitated protein biomarkers with a solution of guanidine hydrochloride.

5. The method of aspect 4, further comprising washing the precipitated protein biomarkers with ethanol.

6. The method of any one of aspects 1-5, wherein the hair segments are arranged in an order corresponding to their position along a length of hair to allow longitudinal sampling.

7. The method of any one of aspects 1-6, wherein the hair sample has a weight of at least 20 mg.

8. The method of aspect 7, wherein the hair sample has a weight ranging from about 20 mg to about 200 mg.

9. The method of aspect 8, wherein the hair sample has a weight ranging from about 50 mg to about 100 mg.

10. The method of any one of aspects 1-9, wherein the method is performed at room temperature.

11. The method of any one of aspects 1-10, wherein the method is performed in under 3 hours.

12. The method of any one of aspects 1-11, further comprising performing chromatography to purify an RNA biomarker or a protein biomarker.

13. The method of aspect 12, wherein the RNA biomarker is a messenger RNA (mRNA), a microRNA (miRNA), a transfer RNA (tRNA), a small nucleolar RNA (snoRNA), or a ribosomal RNA (rRNA).

14. The method of aspect 12 or 13, wherein said measuring the level of the RNA biomarker comprises performing a hybridization-based method, a polymerase chain reaction (PCR)-based method, or a nucleic acid sequencing method.

15. The method of aspect 14, wherein said measuring the level of the RNA biomarker comprises performing microarray analysis, reverse transcriptase polymerase chain reaction (RT-PCR), Northern blotting, RNA-Seq, or serial analysis of gene expression (SAGE).

16. The method of aspect 12, wherein the protein biomarker is a non-structural hair protein.

17. The method of aspect 16, wherein the protein biomarker is a hormone, a receptor, a transcription factor, a transporter, an enzyme, or a growth factor.

18. The method of aspect 17, wherein the hormone is oxytocin.

19. The method of any one of aspects 16-18, wherein said measuring the level of the protein biomarker comprises performing mass spectrometry, or tandem mass spectrometry, an enzymatic or biochemical assay, liquid chromatography, NMR, an enzyme-linked immunosorbent assay (ELISA), a radioimmunoassay (RIA), an immunofluorescent assay (IFA), fluorescence-activated cell sorting (FACS), or a Western Blot.

20. The method of any one of aspects 1-19, wherein a detergent is not used in the method.

21. The method of any one of aspects 1-20, further comprising comparing said levels of the RNA and protein biomarkers to reference levels of the biomarkers to determine a diagnosis or a prognosis for a subject.

22. The method of aspect 21, wherein the subject has not yet developed clinical symptoms.

23. The method of aspect 21, wherein the subject has developed clinical symptoms.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

Methodology to Measure Non-Structural Proteins and RNA species from Human Hair

### Introduction

No methods are currently available for the holistic monitoring of long-term health, aging-related health decline, communicable or non-communicable diseases, or any other chronic health conditions. Longitudinal sampling of hair to measure non-structural proteins and microRNAs will reveal unique biomarker profiles for wellness or disease that are not currently available. This multiomic approach is a solution to several critical gaps that exist in indexing the health status of humans at all ages, to reliably differentiate wellness versus illness, pathogenesis versus salutogenesis. Multiple other groups are trying to close these critical gaps by applying multiomic methodologies to serial blood samples that are obtained over several months or up to 2 years, or from the longitudinal sampling of other biofluids (urine, saliva, mucosal secretions, cerebrospinal fluid, or other tissue fluids). Measuring biomarkers in hair may identify individuals at risk for poor health outcomes, enabling early intervention, avoidance of disease progression, and markedly reduced healthcare costs.

### Non-Structural Hair Protein Extraction Method

Finely chopped hair (10 mg) was incubated in 500 μl Tri-Reagent (Molecular Research Center, Inc; Fisher Scientific) at room temperature for 10 minutes, followed by 100 μl chloroform (shaken vigorously for 15 seconds) and stored at room temperature for 10 minutes. Samples were centrifuged at, 12000 g, 4° C. for 10 minutes to separate the aqueous RNA fraction. To sediment protein from the organic phase, samples were incubated with 1.5 ml acetone at 30° C. for 1 hour, followed by centrifugation at 8000 g for 5 minutes. The supernatant was removed, the sediment suspended in 1 ml, 0.3 M guanidine hydrochloride/95% ethanol/2.5% glycerol, stored for 10 minutes at room temperature, followed by centrifugation at 8000 g for 5 minutes. The supernatant was decanted, and the wash was repeated one additional time. Finally, the protein pellet was washed 2 times in 95% ethanol (without glycerol), then centrifuged for 5 minutes at 8000 g and 4° C. The protein pellet was stored frozen under fresh 95% ethanol, −20° C. until reconstituted according to the proteomics method described in the following paragraph.

### Proteomics Methods

Samples were brought to room temperature, followed by centrifugation at 8000 g for 5 minutes. Ethanol was decanted, and the sample was dried under a spin-vac. Protein pellets were resuspended in 50 mM ammonium bicarbonate in the presence of 0.0015% ProteaseMAX (Promega), and the total protein amount was estimated with a Pierce BCA protein assay. Proteins were digested with 0.25 μg of Trypsin/LysC (Promega) at a 1:100 enzyme/substrate ratio overnight at 37° C. Proteolytic digestion was quenched with 1% formic acid; peptides were dried by speed vac before dissolving in 30 μl of reconstitution buffer (2% acetonitrile+0.1% Formic acid) to a concentration of 1 μg/μl; 2 μl of this solution was injected into the mass spectrometry instrument. Mass spectrometry experiments were performed on Orbitrap Fusion Tribrid mass spectrometer with liquid chromatography using nanoACQUITY ultra performance liquid chromatography. For a typical Liquid Chromatography/Mass Spectrometry experiment, a flow rate of 450 nL/min was used, where mobile phase A is 0.2% formic acid in water and mobile phase B is 0.2% formic acid in acetonitrile. Analytical columns were pulled and packed in-house using fused silica with an I.D. of 100 microns packed with Magic 1.8 micron 120 Å UChrom C18 stationary phase (nanoLCMS Solutions) to a length of ˜25 cm. Peptides were directly injected onto the analytical column using a gradient (2-45% B, followed by a high-B wash) of 80 minutes. The mass spectrometer was operated in data-dependent fashion using collision induced dissociation for generating the MS/MS spectra, collected in the ion trap with a collisional energy setting of 35.

The *.RAW data files were processed using Byonic v3.6.0 software to identify peptides and infer protein isoforms using the Uniprot Homo sapiens database. Proteolysis with Trypsin/LysC was assumed to be semi-specific allowing for N-ragged cleavage with up to two missed cleavage sites. Precursor mass accuracies were held within 12 ppm and 0.4 Da for MS/MS fragments. Proteins were held to a False Discovery Rate (FDR) of 1% or lower, using standard target-decoy approaches. To avoid spurious results, we selected proteins that were observed with >3 spectral counts for further data processing.

Innovative features of our method include:
1. Application of a Multiomics Approach to the analysis of Human Hair.
2. Use of a single sample of hair to provide time-series information about the protein expression, metabolic regulation, and gene expression ongoing in the human body.
3. Use of a Non-Invasive, Pain-Free Method that simplifies sample handling and allows for sample processing and storage at room temperature. Serial segmentation of the hair sample to assess sequential epochs of protein expression, metabolic regulation, and gene expression across different time periods.
4. Specific Analytical Advances are detailed in the three Principles noted below:

Principle 1: Simultaneous collection of proteins and RNA species from human hair (concurrent sampling in a sequential series that incorporates harvesting multiple types of RNA followed by harvesting hair proteins); this has never been done before using human hair samples.

Principle 2: Steps to maximize the yield of RNA species using TRI-reagent by for example:
Increase individual hair sample to 50-100 mg of hair weight
Add chloroform to dissipate the lipid ring containing RNA into the aqueous phase
Stringent sample preparation to generate a minimum of 2 mcg RNA from each sample

Principle 3: Purification of the non-structural proteins and elimination of the structural proteins in hair, by, for example:
Applying TRI-reagent, a monophasic solution of phenol and guanidinium isothiocyanate, for the first time ever to extract the protein components of human hair
Use of Acetone to precipitate hair proteins (vs. alcohols and other reagents in other methods)
Removal of reagents that are expected to solubilize the structural proteins (in our extraction process, the structural proteins are treated as contaminants)
Removal of debris containing the highly cross-linked structural hair proteins
Reduced number of protein pellet washes using Guanidine Hydrochloride (to remove the components of TRI-reagent)
Avoid all detergents during pellet solubilization, (SDS, SDS+TritonX, urea, tris or PBS, DDT)
No addition of Glycerol to prevent the drying extracted hair proteins
Addition of 2 washes with Ethanol (100%) and storage of protein pellet in 100% Ethanol
Performance of all extraction steps at Room Temperature (other methods use extraction steps at 50 to 70 degrees Celsius, which denatures and degrades the non-structural proteins in hair)
Our unique hair extraction process also reduces the hair sample processing time from 3-4 days in other methods to less than 3 hours with our method.

What is claimed is:

1. A method of measuring one or more biomarkers in a hair sample, the method comprising:
(a) obtaining a hair sample from a subject;
(b) segmenting the hair sample to produce a sequential series of hair segments;
(c) isolating RNA biomarkers and protein biomarkers from one or more of the hair segments;
(d) removing structural hair proteins; and
(e) measuring levels of the isolated RNA biomarkers and protein biomarkers.

2. The method of claim 1, wherein said isolating RNA biomarkers and protein biomarkers comprises:
(a) mixing the one or more hair segments with a monophasic solution of phenol and guanidinium isothiocyanate;
(b) adding chloroform to the monophasic solution;
(c) centrifuging the monophasic solution, wherein the monophasic solution is separated into an aqueous phase and an organic phase;
(d) isolating protein biomarkers from the organic phase; and
(e) isolating RNA biomarkers from the aqueous phase.

3. The method of claim 2, further comprising adding acetone to the organic phase to precipitate the protein biomarkers in the organic phase.

4. The method of claim 3, further comprising washing the precipitated protein biomarkers with a solution of guanidine hydrochloride.

5. The method of claim 4, further comprising washing the precipitated protein biomarkers with ethanol.

6. The method of any one of claims 1-5, wherein the hair segments are arranged in an order corresponding to their position along a length of hair to allow longitudinal sampling.

7. The method of any one of claims 1-6, wherein the hair sample has a weight of at least 20 mg.

8. The method of claim 7, wherein the hair sample has a weight ranging from about 20 mg to about 200 mg.

9. The method of claim 8, wherein the hair sample has a weight ranging from about 50 mg to about 100 mg.

10. The method of any one of claims 1-9, wherein the method is performed at room temperature.

11. The method of any one of claims 1-10, wherein the method is performed in under 3 hours.

12. The method of any one of claims 1-11, further comprising performing chromatography to purify an RNA biomarker or a protein biomarker.

13. The method of claim 12, wherein the RNA biomarker is a messenger RNA (mRNA), a microRNA (miRNA), a transfer RNA (tRNA), a small nucleolar RNA (snoRNA), or a ribosomal RNA (rRNA).

14. The method of claim 12 or 13, wherein said measuring the level of the RNA biomarker comprises performing a hybridization-based method, a polymerase chain reaction (PCR)-based method, or a nucleic acid sequencing method.

15. The method of claim 14, wherein said measuring the level of the RNA biomarker comprises performing microarray analysis, reverse transcriptase polymerase chain reaction (RT-PCR), Northern blotting, RNA-Seq, or serial analysis of gene expression (SAGE).

16. The method of claim 12, wherein the protein biomarker is a non-structural hair protein.

17. The method of claim 16, wherein the protein biomarker is a hormone, a receptor, a transcription factor, a transporter, an enzyme, or a growth factor.

18. The method of claim 17, wherein the hormone is oxytocin.

19. The method of any one of claims 16-18, wherein said measuring the level of the protein biomarker comprises performing mass spectrometry or tandem mass spectrometry, an enzymatic or biochemical assay, liquid chromatography, NMR, an enzyme-linked immunosorbent assay (ELISA), a radioimmunoassay (RIA), an immunofluorescent assay (IFA), fluorescence-activated cell sorting (FACS), or a Western Blot.

20. The method of any one of claims 1-19, wherein a detergent is not used in the method.

21. The method of any one of claims 1-20, further comprising comparing said levels of the RNA and protein biomarkers to reference levels of the biomarkers to determine a diagnosis or a prognosis for a subject.

22. The method of claim 21, wherein the subject has not yet developed clinical symptoms.

23. The method of claim 21, wherein the subject has developed clinical symptoms.
